(19) United States
(12) Patent Application Publication
(10) Pub. No.: US 2015/0314063 A1
(43) Pub. Date: Nov. 5, 2015

(54) DELIVERY OF A THERAPEUTIC FLUID

(71) Applicant: Insuline Medical Ltd., Petach-tikva (IL)

(72) Inventors: Ron NAGAR, Tel Aviv-yafo (IL); Gabriel BITTON, Jerusalem (IL)

(21) Appl. No.: 14/650,554

(22) PCT Filed: Dec. 6, 2013
(86) PCT No.: PCT/IB2013/003120

§ 371 Date: Jun. 8, 2015

Related U.S. Application Data
(60) Provisional application No. 61/734,536, filed on Dec. 7, 2012.

(51) Int. Cl. A61M 5/142; A61M 5/172; A61M 5/14

(52) U.S. Cl. CPC A61M 5/14248; A61M 5/14244; A61M 5/1413; A61M 5/172; A61M 2205/3606; A61M 2202/0007; A61M 2205/05; A61M 2205/3569; A61M 2205/3693

(57) ABSTRACT

Systems, methods and devices for delivering a therapeutic fluid into tissue are disclosed. The system includes an infusion set having a catheter for delivery of a dose of therapeutic fluid into a tissue via an infusion tube, a treatment element that applies treatment to the tissue proximate to the catheter, a catheter adaptor having a first transponder, a pump adaptor, in communication with the catheter adaptor, having a second transponder that communicates with the first transponder, and an infusion detection sensor that detects an infusion of the therapeutic fluid. Upon detection of an infusion of the therapeutic fluid by the infusion detection sensor, the second transponder communicates a signal indicative of the detected infusion to the first transponder causing the catheter adaptor to apply treatment using the treatment element. At least one of a strength and a duration of the treatment corresponds to a dose of the infused therapeutic fluid.

### CROSS-REFERENCE TO RELATED APPLICATIONS

This application claims priority to U.S. Provisional Patent Application No. 61/734,536, filed Dec. 7, 2012, and entitled “Wireless Insupatch System and Method,” the disclosure of which is incorporated herein by reference in its entirety.

### TECHNICAL FIELD

Some embodiments of the present disclosure generally relate to the administration of therapeutic fluids, and in particular, to the administration of therapeutic fluids infused subcutaneously using a therapeutic fluid infusion system and/or a therapeutic fluid delivery system into a tissue of a patient.

### BACKGROUND

Diabetes is a very serious illness affecting millions of people today. Many diabetic patients require injection of therapeutic fluids, such as insulin, to maintain proper levels of glucose in their blood in order to survive. Such injections of insulin are performed using drug delivery systems.

Many conventional medical treatment systems use drug delivery systems that employ subcutaneous infusions of therapeutic fluids, including drugs, proteins, and other compounds. These delivery systems, such as in the area of insulin administration, use subcutaneous catheters and continuous subcutaneous insulin infusion (“CSII”) pumps. Conventional insulin pumps are attached to a disposable thin plastic tube or a catheter through which insulin passes into the tissue. The catheter can typically be inserted transcutaneously on the patient's abdomen and may be replaced every two to three days.

Other types of insulin pumps (such as, the OMNIPOD® pump manufactured by Insulet Corporation, Bedford, Mass., USA) do not have an external catheter and, instead, include a catheter port that is embedded into the pump mechanism.

In many instances, the patients may require insulin delivery around the clock to keep proper levels of glucose in their blood. Insulin can be delivered at a basal rate or in bolus doses. The basal rate represents a continuous delivery of a particular amount of insulin to the patient. Such continuous delivery of insulin keeps patient's blood glucose in the desired range between meals and overnight. The bolus dose is an amount of insulin delivered to the patient matching a dose of carbohydrates consumed by the patient. When patient consumes food, his or her level of glucose typically rises. Some conventional pump mechanisms are configured to react upon command, or by way of an algorithm, to the increase in glucose levels by delivering a bolus dose of insulin that matches the rise in the level of glucose and prevents large glucose excursions. However, many conventional subcutaneous drug delivery systems are incapable of quickly matching or preventing the rise of blood glucose. The delay in such matching is also true in case of “rapid-acting” insulin. Some of the reasons for this delay include a lag in the absorption of insulin from the infusion site or infusion location and the time it takes for complex insulin molecules to break down into monomers.

Additionally, since blood glucose levels rise immediately following the meal, the delay in matching insulin to the rising levels may cause post prandial hyperglycemic events (i.e., when levels of blood glucose are above normal) to occur. Further, occasionally after a certain period of time passes (e.g., 2-3 hours) after a meal, the blood glucose levels drop, yet insulin concentrations in the blood rise. The rise may be followed by the peak of the systemic insulin effect and result in causing hypoglycemic events (i.e., when levels of blood glucose are below normal) to occur. Both hyperglycemic and hypoglycemic events are undesirable.

At an insulin infusion or any therapeutic fluid or drug infusion location, there may be large variations in the local blood perfusion, depending on the ambient temperature, physiological parameters and other parameters. This may induce large variations to the delay of the peak of time profile of the insulin or drug action. Those variations in the insulin or drug peak action further increase the variability in the blood glucose level within the patient. The insulin or drug peak action may comprise the time when the insulin or drug reaches its maximal effect on a treatment target. The peak of time profile may be the time spanning from delivery of the insulin or drug to the patient until the insulin or drug reaches its peak action. As such, there is a need for a system that is capable of providing efficient and timely delivery of therapeutic fluid (e.g., insulin, drug, etc.) while reducing variations in the therapeutic fluid peak action, decreasing variability in the blood glucose level in the patient, as well as providing other beneficial effects.

SUMMARY OF DISCLOSURE

In some embodiments, the current subject matter relates to a system and a method that can provide efficient and timely delivery of a therapeutic fluid to the patient.

In some embodiments, the current subject matter relates to a system and a method for delivering insulin to the patient that can improve effectiveness of insulin in the blood to maintain normal levels of blood glucose and prevent or reduce hyperglycemic and hypoglycemic events.

In some embodiments, a therapeutic fluid delivery system or therapeutic fluid infusion system is provided. The system can include an infusion pump adaptor and a catheter adaptor. The infusion pump adaptor can include an infusion pump adaptor housing that can contain a bolus detection element, a wireless transponder, a power source (e.g., a rechargeable battery, a one-time use battery and/or any other power source), a controller, as well as various electrical circuitry. The catheter adaptor can include a catheter adaptor housing that can contain a wireless transponder, a power source (e.g., a rechargeable battery, a one-time use battery and/or any other power source), a controller, a thermistor, and various electrical circuitry. The catheter adaptor can be configured to couple to an infusion set containing a catheter. The infusion set can include an adhesive for coupling the infusion set to an infusion location on the patient's body where the drug infusion will take place, a treatment element (e.g., a heating element) for application of treatment to the patient either before, during, and/or after infusion of the drug, a catheter, an infusion tube coupled to a drug reservoir from which the drug is being delivered to the patient.

In some embodiments, the treatment element can be embedded into a base of the infusion set. The base can include an adhesive layer which can affix the infusion set and the catheter to the infusion location on the patient. The catheter adaptor can be configured to be mechanically affixed to the infusion set (e.g., using fix and release elements, and/or any other way). The catheter adaptor can also include a wireless transponder, a rechargeable power source, thermistor, electrical circuitry, electrical contacts, and/or various other elements. When the catheter adaptor is connected to the catheter, the electrical contacts of the catheter adaptor can electrically contact the catheter electrical contacts to conduct current to the treatment element and, at the same time, can conduct heat back to a thermistor which can be disposed within the electrical circuit of the catheter adaptor.

In some embodiments, the current subject matter relates to a system for delivering a therapeutic fluid into tissue which additionally can include one or more of the following features:
an infusion set comprising a catheter for delivery of a dose of therapeutic fluid into tissue via an infusion tube;
a treatment element configured to apply a treatment to tissue proximate the catheter;
a catheter adaptor comprising a first transponder; and
a pump adaptor in communication with the catheter adaptor and comprising a second transponder configured to communicate with the first transponder and an infusion detection sensor configured to detect an infusion of the therapeutic fluid;
wherein, upon detection of an infusion of the therapeutic fluid by the infusion detection sensor, the second transponder can be configured to communicate a signal indicative of the detected infusion to the first transponder, wherein the catheter adaptor is configured to cause the treatment element to apply treatment to the tissue proximate to the catheter based on at least one of the following: the signal indicative of the detected infusion and a manual activation (such as by using a button) of the catheter adaptor causing the treatment element to apply treatment causing the catheter adaptor to apply treatment via the treatment element to tissue proximate the catheter,
wherein at least one of a strength and a duration of the treatment can correspond to the dose.

In some embodiments, the treatment element can apply treatment during at least one of the following times: before infusion of the therapeutic fluid, during infusion of the therapeutic fluid, and after infusion of the therapeutic fluid.

At least one of the first transponder and the second transponder can include at least one of the following: a wireless transponder, and a radio frequency identification device (“RFID”), an antenna, a transducer, and/or an RLC circuit, an electrical circuit for analog or digital short range communication, or a digital communication mode, including WIFI or BLUETOOTH®.

The pump adaptor can be connected to a pump and a reservoir containing the therapeutic fluid. The pump can be configured to pump the therapeutic fluid from the reservoir to the catheter via the infusion tube.

In some embodiments, the infusion detection sensor can be configured to detect an amount of the therapeutic fluid being pumped by the pump based on at least one movement of the pump. In some embodiments, the dose of the therapeutic fluid being infused can be a bolus dose. In some embodiments, the dose of the therapeutic fluid being infused can correspond to a basal rate. In some embodiments, the dose of the therapeutic fluid being infused can be at or above a predetermined dose.

In some embodiments, the therapeutic fluid can include at least one of the following: subcutaneously delivered therapeutic fluids, insulin, rapid-acting insulin, insulin mimetics, insulin analogs, and/or any other types of insulin, pain relief drugs or cancer treatment drugs. The pump adaptor can include a pump adaptor power source and a pump adaptor controller, wherein the controller, upon receiving an indication from the infusion detection sensor, can instruct the second transponder to communicate with the first transponder.

In some embodiments, the catheter adaptor can include a catheter adaptor power source, a catheter adaptor controller. At least one first electrical contact can configured to be coupled to at least one second electrical contact disposed on the infusion set. The catheter adaptor controller can be configured to process the communication received from the second transponder and generate an instruction to the treatment element to initiate the application of treatment. The generated instruction can be provided to the treatment element using the first electrical contact and the second electrical contact.

In some embodiments, the application of treatment can include at least one of the following: heating, cooling, mechanical vibrations, suction, massaging, acoustic stimulation, electromagnetic radiation, magnetic stimulation, radio frequency irradiation, microwave irradiation, electrical stimulation, Transcutaneous Electrical Nerve Stimulation (“TENS”), an additional substance, drugs, medicament, chemicals, biologically active bacteria, biologically inactive bacteria and/or any combination thereof.

In some embodiments, the infusion set can include an adhesive element configured to attach the infusion set to the body. The application of treatment can be configured to modify pharmacokinetic and/or pharmacodynamics profile of the therapeutic fluid being infused.

In some embodiments, the current subject matter relates to a method for delivering a therapeutic fluid into a tissue. The method can include one or more of the following: providing the system for delivering a therapeutic fluid into tissue, initiating an infusion of the therapeutic fluid using an infusion pump, detecting the initiation of the infusion of the therapeutic fluid using the infusion detection sensor, generating a communication to the first transponder using the second transponder, where the communication comprises a signal indicative of the detected infusion, and activating the treatment element using the catheter adaptor to apply treatment in accordance with a dose of the therapeutic fluid being infused.

In some embodiments, initiation of the infusion can include pumping the therapeutic fluid from the reservoir to the catheter via the infusion tube. The detection of the initiation of the infusion can include detecting an amount of the therapeutic fluid being pumped by the pump based on at least one movement of the pump.

In some embodiments, the current subject matter relates to a system for delivering a therapeutic fluid into tissue. The system can include one or more of the following: an infusion set comprising a catheter for delivery of a dose of therapeutic fluid into tissue via an infusion tube, a treatment element configured to apply a treatment to tissue proximate the catheter, a catheter adaptor comprising a transponder, and an infusion detection sensor configured to detect an infusion of the therapeutic fluid. Upon detection of an infusion of the therapeutic fluid by the infusion detection sensor, the transponder can be configured to communicate a signal indicative of the detected infusion. This can cause the catheter adaptor to apply treatment via the treatment element to tissue proximate the catheter. At least one of a strength and a duration of the treatment can correspond to the dose. Alternatively or in addition to, upon activation of a button, the catheter adaptor can be configured to apply treatment via the treatment element to tissue proximate the catheter.

In some embodiments, the current subject matter can implement a tangibly embodied machine-readable medium embodying instructions that, when performed, cause one or more machines (e.g., computers, etc.) to result in operations described herein. Similarly, computer systems are also described that can include a processor and a memory coupled to the processor. The memory can include one or more programs that cause the processor to perform one or more of the operations described herein. Additionally, computer systems may include additional specialized processing units that are able to apply a single instruction to multiple data points in parallel. Such units include but are not limited to so-called “Graphics Processing Units (GPU).”

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The principles and operations of the systems, apparatuses and methods according to some embodiments of the present disclosure may be better understood with reference to the drawings, and the following description. The drawings are given for illustrative purposes only and are not meant to be limiting.

FIG. 1 is a schematic illustration of an exemplary therapeutic fluid delivery system, according to some embodiments of the present disclosure;

FIG. 2 is a schematic illustration of an exemplary therapeutic fluid delivery system, according to some embodiments of the present disclosure;

FIG. 3 is a schematic illustration of an exemplary therapeutic fluid delivery system, according to some embodiments of the present disclosure;

FIG. 4 is a schematic illustration of an exemplary therapeutic fluid delivery system, according to some embodiments of the present disclosure; and

FIG. 5 is a schematic illustration of an exemplary therapeutic fluid delivery system, according to some embodiments of the present disclosure.

### DETAILED DESCRIPTION

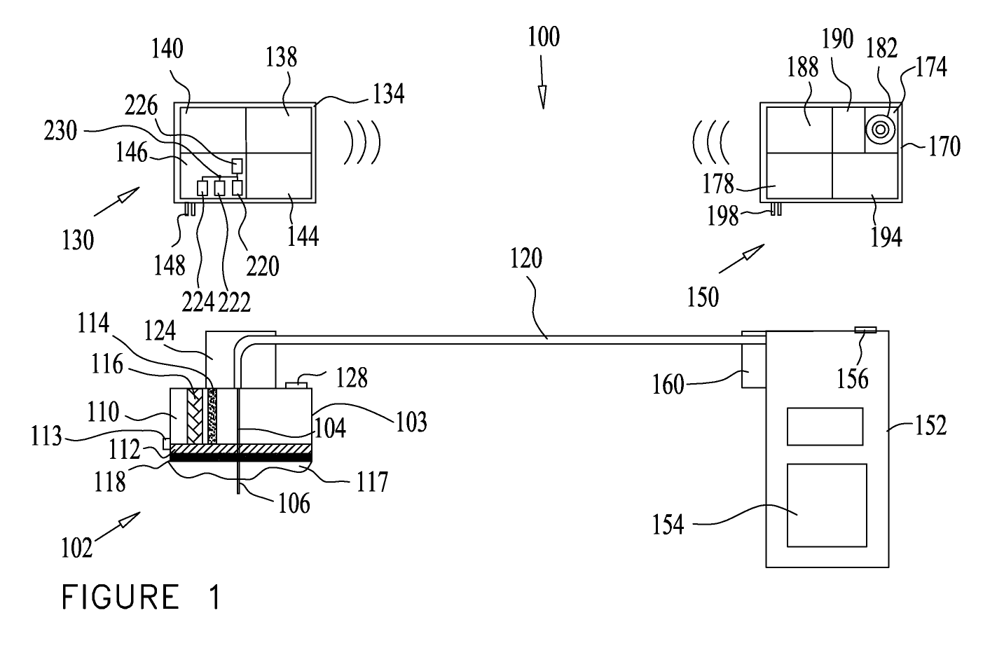

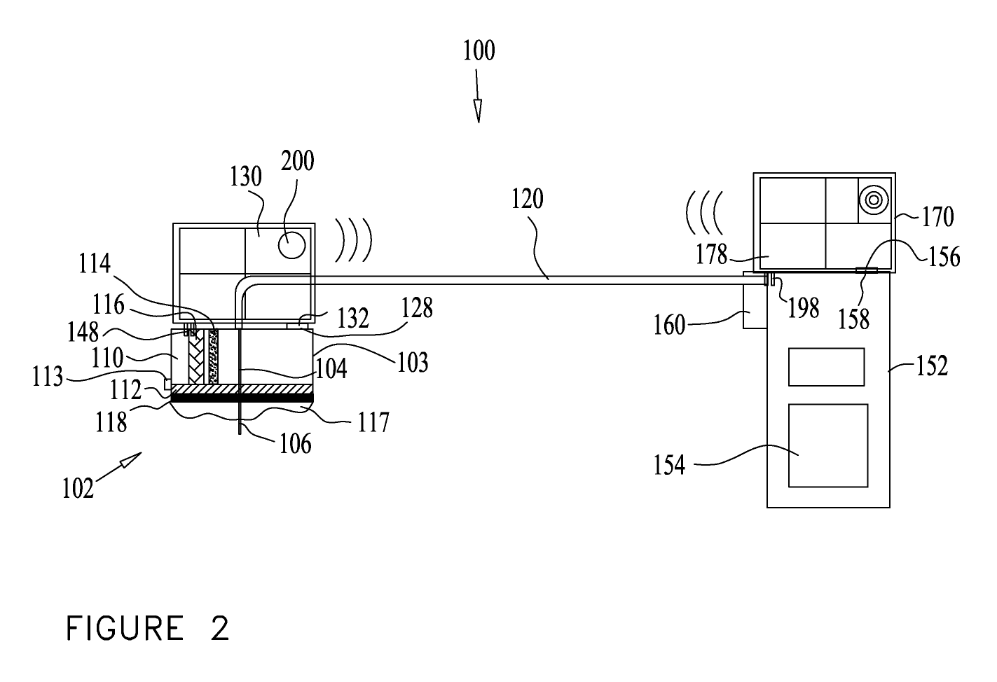

FIGS. 1 and 2 illustrate an exemplary system 100 for delivering a therapeutic fluid into a tissue, according to some embodiments of the present disclosure. The therapeutic fluid delivery system 100 can include an infusion set 102, which is coupled to an infusion pump 152 using an infusion tube 120, a catheter adaptor 130, and a pump adaptor 150. The infusion set 102 is configured to be placed on a tissue 117 for delivery of the therapeutic fluid. The catheter adaptor 130 is configured to be coupled to the infusion set 102. The catheter adaptor 130 is further configured to communicate with the pump adaptor 150, as will be discussed below. The pump adaptor 150 is configured to be coupled to the infusion pump 152.

The infusion set 102 can include an infusion set housing 103 for housing a catheter 104 provided with a needle 106. The catheter 104 can be placed in a catheter base 110, which can include a treatment element 112.

In some embodiments, the treatment element 112 can be placed on the catheter 104 or in proximity thereto. In some embodiments, the treatment element 112 can be mechanically attached to the catheter 104. In some embodiments, the treatment element 112 can be placed in proximity to the infusion location yet without physical contact with the catheter 104.

In some embodiments, the treatment element 112 can be configured to apply a treatment to tissue 117 proximate the catheter 104. In some embodiments, the treatment element can be configured to apply any suitable treatment capable of modifying the pharmacokinetic and/or pharmacodynamics profile of the therapeutic fluid being infused. In some embodiments, the treatment element can be also configured to apply any suitable treatment capable of enhancing a tissue response to the delivered therapeutic fluid.

The treatment can include, but not limited to, for example, any one of: heating, cooling, mechanical vibrations, suction, massaging, acoustic stimulation (e.g., ultrasound), electromagnetic radiation, electric field stimulation, magnetic field stimulation, radio frequency irradiation, microwave irradiation, electrical stimulation, magnetic stimulation, Transcutaneous Electrical Nerve Stimulation (“TENS”), or the like, and/or any combination of the above treatments to improve the drug's pharmacokinetic profile and/or pharmacodynamic profile. In some embodiments, the treatment element 112 can stimulate or inhibit tissue by introducing additional substances (in addition to the therapeutic fluid), for example, including, but not limited to, drugs, medicament, chemicals, biologically active bacteria, biologically inactive bacteria or the like or also any combination of the above treatments to improve the drug's pharmacokinetic profile and/or pharmacodynamic profile.

In some embodiments, the applied treatment can reduce variability of the therapeutic fluid absorption in the blood and/or lymph system and/or its local and/or systemic effects. For example, heating the tissue location in the infused location to a preset regulated temperature before, during and/or after the therapeutic fluid infusion and absorption into the blood, can make local blood perfusion at the infused location more reproducible and the therapeutic fluid absorption process more uniform and reproducible as well. Also, by reducing the delay between the therapeutic fluid delivery into the tissue and absorption into the blood system, the variability of the therapeutic fluid action induced by the delayed peak action profile can be reduced.

In some embodiments, the treatment element 112 can be triggered manually by the user or by any other means. The user can activate the treatment element 112 either before, during and/or after the infusion of the therapeutic fluid from the infusion pump 152. In such embodiments, the activation of treatment element 112 can be performed by pressing a button 113 located on the infusion set 102 (or a sequence of buttons for activating the treatment element 112).

In some embodiments, the treatment element 112 can be placed outside of the catheter base 110 in any suitable location for modifying the pharmacokinetic and/or pharmacodynamics profile of the therapeutic fluid being infused.

In some embodiments, the treatment element 112 can be configured to apply a selected treatment based on the dosage(s) of the therapeutic fluid being administered. The treatment can be applied for a predetermined period of time, on a predetermined schedule, at a predetermined time of day, as desired by the user, and/or based on any other pattern, and/or any combination thereof.

In some embodiments, the treatment element 112 can apply treatment either before, during, and/or after infusion of the therapeutic fluid to the subcutaneous tissue of the patient. The treatment can be applied continuously, intermittently, periodically, cyclically and/or in any other manner.

In some embodiments, the catheter base 110 can include electrical contact(s) 114 connected to the treatment element 112 for operation thereof. In some embodiments, in addition to the electrical contact(s) 114 or in place thereof, other elements for operation of the treatment element 112 can be included. Such elements can include various circuitry, power source(s), and/or any other electro-mechanical components. The electrical contact(s) 114 can be configured to conduct current to the treatment element 112 from a power source (not shown in FIG. 1). In some embodiments, where the treatment element 112 applies heat, heating conducts 116 can be provided for causing the treatment element 112 to apply heat to the subcutaneous tissue.

Prior to the delivery of the therapeutic fluid, the infusion set 102 can be configured to be coupled to a predetermined infusion tissue location 117 on the patient using an adhesive element 118 that can be disposed on the bottom of the infusion set 102.

The infusion tube 120 can extend from catheter 104 and can be connected to the infusion set 102 using an infusion set catheter connector 124. The infusion tube 120 can be configured for delivery of a dose of the therapeutic fluid to the catheter 104 and can be connected to the catheter 104 in any suitable manner. As shown in FIG. 1, the infusion tube 120 can be coupled to the infusion pump 152, from which the therapeutic fluid can be transported to the catheter 104 via the infusion tube 120.

In some embodiments, a connection port 128 can be provided for connecting the infusion set 102 to the catheter adaptor 130. In some embodiments, as shown in FIG. 2, the connection port 128 can include a mechanical connector mechanism 132 for mechanically connecting the infusion set 102 to the catheter adaptor 130. The mechanical connector mechanism 128 can include a mechanical fix and release mechanism and can protrude from catheter base 110 or can include any other mechanism for connecting the infusion set 102 to the catheter adaptor 130.

In some embodiments, the catheter adaptor 130 can be connected to the infusion set 102 in any suitable manner, such as electro-mechanically, electrically and/or magnetically, for example.

Referring back to FIG. 1, the catheter adaptor 130 can include a catheter adaptor housing 134 for housing a first transponder 138 and a power source 140, an electrical circuitry 144, and a catheter adaptor controller 146. The catheter adaptor controller 146 can be configured to control the operation of the treatment element 112 and can be connected thereto via electrical contacts 148 (or first electrical contacts 148), which can connect with electrical contact(s) 114 (or second electrical contacts 114).

In some embodiments, the electrical contacts 148 can connect with electrical contact(s) 114 in any suitable manner, such as electrically and/or electro-mechanically, for example.

The catheter adaptor 130 can include additional elements for operation of the treatment element 112, such as a thermistor (not shown in FIG. 1), for example, and/or any other suitable element(s).

In some embodiments, the treatment element 112 can include a heater. A thermistor can be provided for measuring a temperature of the heat applied by the treatment element 112 and can be used to control the temperature so it is maintained within a predetermined temperature range. In some embodiments, the heating temperature can be controlled so as not to exceed a limiting temperature sustainable by the therapeutic fluid so as to prevent degradation or overheating thereof. In some embodiments, a maximum limiting temperature can be calibrated for each therapeutic fluid and/or class of therapeutic fluids. In some embodiments, the application of heat can be controlled so that the temperature of the drug being infused, the subcutaneous tissue, and/or any other parameters associated with the drug delivery are sustained within a predetermined range. By way of a non-limiting example (where the therapeutic fluid can be some type of insulin), the limiting temperature can be approximately 37° Celsius.

In some embodiments, the power source 140 can be any suitable power source such as a rechargeable battery, for example.

In some embodiments, the catheter adaptor 130 can communicate with the infusion pump adaptor 150 in any suitable manner, as described below.

In some embodiments, the infusion pump adaptor 150 can be connected to the infusion pump 152 and a therapeutic fluid reservoir 154, disposed in infusion pump 152, in any suitable manner, such as via a pump connection port 156. In some embodiments, as shown in FIG. 2, the pump connection port 156 can include a mechanical connector mechanism 158 for mechanically connecting the infusion pump adaptor 150 to the infusion pump 152. The mechanical connector mechanism 158 can include a mechanical fix and release mechanism and can protrude from infusion pump 152 or can include any other mechanism for connecting the infusion pump adaptor 150 to the infusion pump 152.

In some embodiments, the infusion pump adaptor 150 can be connected to the infusion pump 152 in any suitable manner, such as electro-mechanically, electrically and/or magnetically, for example.

In some embodiments, the therapeutic fluid reservoir 154 can be disposed within the infusion pump 152 and/or any other element that can be used for delivery of a therapeutic fluid into the subcutaneous tissue of the patient.

In some embodiments, the therapeutic fluid can include any therapeutic fluid delivered to the subcutaneous tissue. In some non-limiting examples, the therapeutic fluid can include at least one of the following: insulin, rapid-acting insulin, insulin mimetics, insulin analogs, and/or any other types of insulin, and/or any other drugs, such as pain relief drugs or cancer treatment drugs and/or any other combinations of drugs.

The infusion tube 120 can be connected to the infusion pump 152, via a pump connector 160 or in any other suitable manner. The infusion pump 152 can be configured to deliver a dose of the therapeutic fluid from the therapeutic fluid reservoir 154, via the infusion tube 120, to the catheter 104 for delivery of the therapeutic fluid into the subcutaneous tissue of the patient.

In some embodiments, the infusion pump 152 can be preset by a user to deliver a basal dose of the therapeutic fluid. The infusion pump 152 can extract the basal dose from the therapeutic fluid reservoir 154. The therapeutic fluid can be delivered via the infusion tube 120 to the infusion set 102 for infusion, and via the catheter 104 (and/or needle 106), into the subcutaneous tissue of the patient at the predetermined infusion tissue location 117.

In some embodiments, the user can be the patient and/or any other individual, including a medical professional, a caregiver, and/or any other person.

In some embodiments, the user can use the system 100 to deliver a bolus dose of the therapeutic fluid. For a bolus dose delivery, prior to infusion, the user can preset the infusion pump 152 to deliver an appropriate bolus dose of the therapeutic fluid.

The pump adaptor 150 can include a pump adaptor housing 170 which can house an infusion detection sensor 174 configured to detect infusion of the therapeutic fluid at the infusion tissue location 117 in any suitable manner. A pump adaptor controller 178 can be provided for processing the signal detected by the infusion detection sensor 174.

In some embodiments, the infusion detection sensor 174 can include a pick-up coil 182, such as shown in FIG. 1. The pick-up coil 182 can detect mechanical, electric and/or electromagnetic signals and/or any other signals generated upon movement of a pump motor (not shown in FIG. 1) of the infusion pump 152 for pumping the therapeutic fluid from the reservoir 154 towards the infusion tube 120. In some embodiments, the pump adaptor controller 178 can be provided to process the signal detected by the pick-up coil 182 for generating information including the dose of the therapeutic fluid and/or duration of the therapeutic fluid delivery.

In some embodiments, the infusion detection sensor 174 can include any element for detecting infusion of the therapeutic fluid, such as the mechanical, electric, electromagnetic and/or acoustic emission of the infusion pump 152, the infusion pump motor or electronics associated with the infusion pump 152 or any other component associated with the infusion pump 152.

In some embodiments, the infusion detection sensor 174 can include any element or sensor for detecting infusion of the therapeutic fluid, such as the flow of the therapeutic fluid delivery in the infusion tube 120.

In some embodiments, the infusion detection sensor 174 can also detect the amount of other information related to the therapeutic fluid delivery, such as the dose, duration, frequency, flow rate and/or temperature of the therapeutic fluid. In some embodiments, where the therapeutic fluid is insulin, the infusion detection sensor 174 can be configured to detect a bolus dose or basal dose being delivered by the infusion pump 152. This signal containing such information can be transmitted to the pump adaptor controller 178 for processing thereof.

In some embodiments, the infusion detection sensor 174 can be configured as a bolus or basal dose detection element. The detection of a bolus dose or basal dose can be performed by detecting the amount of movements during a predetermined time period of the pump motor pumping the therapeutic fluid from the reservoir 154 towards the infusion tube 120. The pump adaptor controller 178 can contain information pertaining to a quantity of therapeutic fluid delivered during each motor movement. Accordingly, the pump adaptor controller 178 can calculate the total quantity of therapeutic fluid delivered, which can constitute the therapeutic fluid dose.

In some embodiments, the pump adaptor 150 can include a second transponder 188 for communication with the first transponder 138.

The first transponder 138 and second transponder 188 can include any elements configured for communication signals, such as receiving signals and for transmission of the signals in any suitable manner.

In some embodiments first transponder 138 and second transponder 188 can include at least one of the following: a wireless transponder, or a radio-frequency identification (“RFID”) device. In some embodiments, any transmission element can be used as the first transponder 138 and second transponder 188. The transmission element can include at least one of the following, for example: a transmitter, a transponder, an antenna, a transducer, and/or an RLC circuit or any suitable components for detecting, processing, storing and/or transmitting a signal, such as electrical circuitry, an analog-to-digital (“A/D”) converter, and/or an electrical circuit for analog or digital short range communication.

In some embodiments, the communication between the catheter adaptor 130 and pump adaptor 150 can be wireless, via an analog short range communication mode, or a digital communication mode including WIFI or BLUETOOTH®, or via a wired connection. Additional examples of such communication can include a network. The network can include a local area network (“LAN”), a wide area network (“WAN”), or a global network, for example. The network can be part of, and/or can include any suitable networking system, such as the Internet, for example, and/or an Intranet. Generally, the term “Internet” may refer to the worldwide collection of networks, gateways, routers, and computers that use Transmission Control Protocol/Internet Protocol (“TCP/IP”) and/or other packet based protocols to communicate therebetween.

The pump adaptor 150 can include a power source 190 which can be any suitable power source such as a rechargeable battery, for example. In some embodiments, the pump adaptor 150 can include electrical circuitry 194 and electrical contacts 198 for operation thereof. As shown in FIG. 2, the pump adaptor 150 is configured to be coupled to the infusion pump 152 (e.g., electrically, mechanically, electro-mechanically, and/or in any other fashion) using the electrical contacts 198.

Referring back to FIG. 1, according to some embodiments, wherein, upon detection of an infusion of the therapeutic fluid by the infusion detection sensor 174, the second transponder 188 can be configured to communicate a signal indicative of the detected infusion to the first transponder 138 causing the catheter adaptor 130, such as by the catheter adaptor controller 146, to apply treatment via the treatment element 112 to tissue proximate the catheter.

In some embodiments, the pump adaptor controller 178, upon receiving an indication from the infusion detection sensor 174, can instruct the second transponder 188 to communicate with the first transponder 138.

In some embodiments, at least one first electrical contact 148 of the catheter adaptor 130 can be configured to be coupled with at least one second electrical contact 114 disposed on the infusion set 102. The catheter adaptor controller 146 can be configured to process the communication received from the second transponder 188 and generate an instruction to the treatment element 112 to initiate the application of treatment, the generated instruction being provided to the treatment element 112 using at least one of the first electrical contact 148 and at least one second electrical contact 114.

In some embodiments, the treatment element 112 can include heating and the catheter adaptor controller 146 can apply the treatment via electrical contacts 148, which can connect with electrical contact(s) 114 and the heating conducts 116 for causing the treatment element 112 to heat the subcutaneous tissue.

In some embodiments, the applied treatment via the treatment element 112 can correspond with the information related to the therapeutic fluid delivery. In some embodiments, at least one of a strength and a duration of the treatment can correspond to a detected therapeutic fluid dose.

In some embodiments, parameters of the applied treatment, applied by the treatment element 112, can correspond with the information related to the therapeutic fluid delivery. These parameters can include at least one of the following: a strength (e.g., when the treatment is heat, the strength can include the temperature of the applied heat), a duration, a type of treatment (e.g., heating, cooling, mechanical vibrations, and/or any other suitable treatment, such as described above), a continuous treatment or cyclic treatment, and/or the frequency of the cyclic treatment.

In some embodiments, the information related to the therapeutic fluid delivery can include information about the dose of the delivered therapeutic fluid.

In some embodiments, the catheter adaptor controller 146 along with the first transponder 138 can monitor and/or detect of signals that can be sent by the second transponder 188 so that the activation of the treatment element 112 can be performed accurately and on a timely basis.

In a non-limiting example, where the infusion detection sensor 174 includes the pick-up coil 182, upon detection of one pump movement per hour, the pump adaptor controller 178 can process the detected signal as a delivery of a basal dose of insulin. The signal can be transmitted by the second transponder 188 to the first transponder 138. Accordingly, the catheter adaptor controller 146 does not cause application of treatment.

By way of a non-limiting example, upon detection of four (4) pump movements per two (2) minutes, the pump adaptor controller 178 can process the signal as a delivery of a bolus dose of insulin. The signal can be transmitted by the second transponder 188 to the first transponder 138. Accordingly, the catheter adaptor controller 146 can cause the treatment element 112 to apply treatment, e.g., by heat at 37° C., continuously for ten (10) minutes.

By way of another non-limiting example, upon detection of twenty (20) pump movements per two (2) minutes, the pump adaptor controller 178 can process the signal as a delivery of a predetermined dose of a selected drug. The signal can be transmitted by the second transponder 188 to the first transponder 138. Accordingly, the catheter adaptor controller 146 can cause the treatment element 112 to apply treatment, e.g., by heat at 41° C., for a duration of fifty (50) minutes, where the heat can be cyclically applied for ten (10) minutes with a five (5) minute interval therebetween.

In some embodiments, the first catheter adaptor controller 146 can be continuously in operation during the time the system 100 is in operation.

In some embodiments, the first transponder 138 can be activated upon receipt of a signal by the catheter adaptor controller 146 that a therapeutic fluid infusion was detected. Upon activation, the first transponder 138 can generate a signal to the second transponder 188 to transmit information relating to the infusion (e.g., dose, strength, duration, etc.) for providing the catheter adaptor controller 146 with the information for determining the parameters (e.g., strength, duration, etc.) of the treatment applied by the treatment element 112.

Detection of the therapeutic fluid infusion can be performed by the infusion detection sensor 174 of the pump adaptor 150. In some embodiments, an infusion detection sensor can be placed out of the pump adaptor 150, such as, in the catheter adaptor 130 and/or in proximity to the catheter 104 and/or the infusion pump 120.

As can be seen, for example in FIG. 2, an infusion detection sensor 200 can be placed in the catheter adaptor 130 and can be configured for detecting infusion of the therapeutic fluid in any suitable manner. For example, the infusion detection sensor 200 can include a flow detector, which can be configured to measure the flow of the therapeutic fluid in the infusion tube or anywhere in proximity to the catheter 104. The catheter adaptor controller 146 can be configured to activate the first transponder 138 upon detection of a predetermined flow rate of the therapeutic fluid. Upon activation, the first transponder 138 can generate a signal to the second transponder 188 to transmit information relating to the infusion (e.g. dose, strength, duration, etc.) for providing the catheter adaptor controller 146 with the information for determining the parameters (e.g. strength, duration, etc.) of the treatment applied by the treatment element 112.

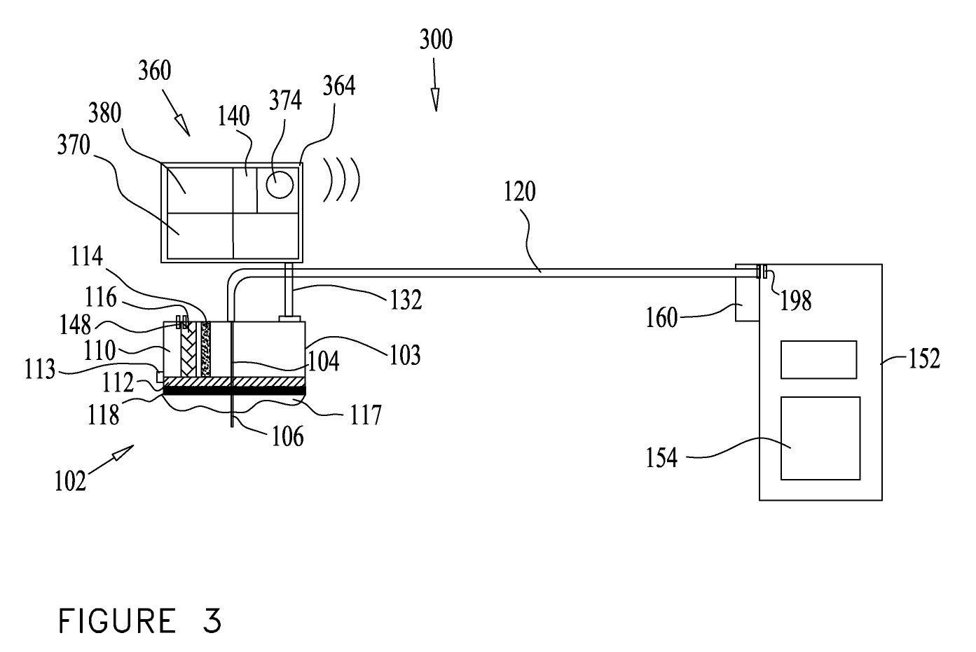

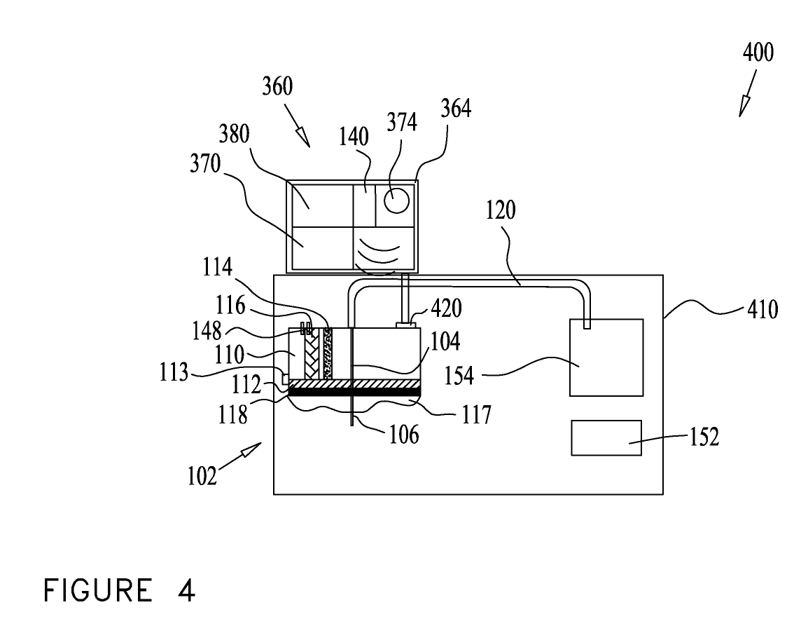

In some embodiments, any one of the catheter adaptor controller 146 and/or the pump adaptor controller 178, adaptor controller 370 (as shown in FIGS. 3 and 4) and/or any other relevant component of the system 100 and/or systems 300 and 400 (as shown in FIGS. 3 and 4) can include a processor, a memory, a storage device, and an input/output device. As shown in FIG. 1, the catheter adaptor controller 146 includes a processor 220, a memory 222, a storage device 224, and an input/output device 226. The processor 220, the memory 222, the storage device 224, and the input/output device 226 can be interconnected therebetween using a system bus 230. The processor 220 can be configured to process instructions for execution within the system 100. In some embodiments, the processor 220 can be a single-threaded processor. In alternate embodiments, the processor 220 can be a multi-threaded processor. The processor 220 can be further configured to process instructions stored in the memory 222 or on the storage device 224, including receiving or sending information through the input/output device 226. The memory 222 can store information within the system 300. In some embodiments, the memory 222 can be a computer-readable medium. In alternate embodiments, the memory 222 can be a volatile memory unit. In some embodiments, the memory 222 can be a non-volatile memory unit.

In some embodiments, the storage device 224 can be capable of providing mass storage for the catheter adaptor controller 146 and/or the pump adaptor controller 178 and/or adaptor controller 370. In some embodiments, the storage device 224 can be a computer-readable medium. In some embodiments, the storage device 224 can be a floppy disk device, a hard disk device, an optical disk device, a tape device, a non-volatile solid state memory, and/or any other type of storage device, and/or any combination thereof. The input/output device 226 can be configured to provide input/output operations for the catheter adaptor controller 146 and/or the pump adaptor controller 178 and/or adaptor controller 370 (as shown in FIGS. 3 and 4). In some embodiments, the input/output device 226 can include a keyboard and/or pointing device. In alternate embodiments, the input/output device 226 can include a display unit for displaying graphical user interfaces.

In some embodiments, the systems 100, 300 or 400 can be configured to detect an infusion of a dose of delivered therapeutic fluid and cause the treatment element to apply treatment to tissue proximate the catheter, where at least one of a strength and a duration of the treatment can correspond to the dose. As described throughout the present disclosure, the detection can be performed by an infusion detection sensor and a controller which can receive a signal from the infusion detection sensor indicating the infusion dose and cause the treatment element to apply the treatment. In some embodiments, the communication between the infusion detection sensor and the controller can be wireless, such as, via first transponder 138 and second transponder 188 (as shown in FIGS. 1 and 2) and/or transponder 380 (as shown in FIGS. 3 and 4). Wireless communication can allow the infusion pump 152 and the infusion set 102 to communicate without using electrical wires. Further, wireless communication can be advantageous as it does not require attaching an electrical wire to the infusion tube 120 for the purposes of providing the above communication. Otherwise, having such an electrical wire can cause obstructions in the flow of the therapeutic fluid through the infusion tube 120. Also, electrical insulation of the infusion tube 120 to prevent heating of and/or conduction by the therapeutic fluid due to the presence of the electrical wire in the vicinity of the tube 120 would not be required. Further, systems 100, 300 and/or 400 can use existing, commercial infusion tube(s) without addition of electrical connection(s).

In some embodiments, as shown in FIGS. 1 and 2, the catheter adaptor 130 can include a power source 140, and the pump adaptor 150 can include a power source 190. In some embodiments, because the power source 190 can be used to power the second transponder 188, the power source 190 can be relatively small. By way of a non-limiting example, the power source 190 can include a battery that can be changed and/or, otherwise, recharged (e.g., infrequently, such as, once a month). This can be advantageous to using a power source in a system having an electrical wire attached to the infusion tube 120, where the power source is used to activate the treatment element 112, thereby requiring the power source to be either changed and/or recharged frequently, such as every three (3) days.

FIG. 3 is a schematic illustration of an exemplary therapeutic fluid delivery system 300, according to some embodiments of the present disclosure. The therapeutic fluid delivery system 300 comprises the infusion set 102, the infusion tube 120 and the infusion pump 152, which were described above and shown in reference to FIGS. 1 and 2. The therapeutic fluid delivery system 300 can include an adaptor 360 which can include components that are similar to components of the catheter adaptor 130 (as shown in FIG. 1), such as the power source 140 and electrical circuitry 144. The adaptor 360 can include a housing 364 for housing an adaptor controller 370. The adaptor controller 370 can be configured to control the operation of the treatment element 112 and can be connected thereto via electrical contacts 148, which can connect with electrical contact(s) 114. The adaptor controller 370 can be configured to process a signal detected by an infusion detection sensor 374. The infusion detection sensor 374 can be configured to detect infusion of the therapeutic fluid at the infusion location 117 in any suitable manner. For example, the infusion detection sensor 374 can operate similar to the infusion detection sensor 200 shown in FIG. 2.

The adaptor controller 370 can be configured to activate a transponder 380 upon detection of a predetermined flow rate of the therapeutic fluid by the infusion detection sensor 374. Upon activation, the transponder 380 can generate a signal indicative of information related to the infusion (e.g., dose, strength, duration, etc.). The signal can be provided to the adaptor controller 370 with the information for determining parameters (e.g., strength, duration, etc.) of the treatment to be applied by the treatment element 112.

In some implementations, the infusion set 102 can be connected to the infusion pump via the infusion tube 120, as shown in FIG. 3.

In some embodiments, the adaptor 360 can include system(s), device(s), and/or component(s) of system(s), device(s) that are disclosed in co-owned International Patent Applications Nos. PCT/IB2009/007600 and/or PCT/IB2012/052335, the disclosures of which are incorporated herein by reference in their entireties.

FIG. 4 is a schematic illustration of an exemplary therapeutic fluid delivery system 400, according to some embodiments of the present disclosure. The therapeutic fluid delivery system 400 can include the infusion set 102, the infusion tube 120, the infusion pump 152 and the adaptor 360 (these components have been described above and shown in FIGS. 1-3. The therapeutic fluid delivery system 400 can include a housing 410 for housing the infusion set 102, the infusion tube 120, and the infusion pump 152.

The adaptor 360 can be mechanically coupled to the housing 410 using a connector 420 and/or using any other suitable means.

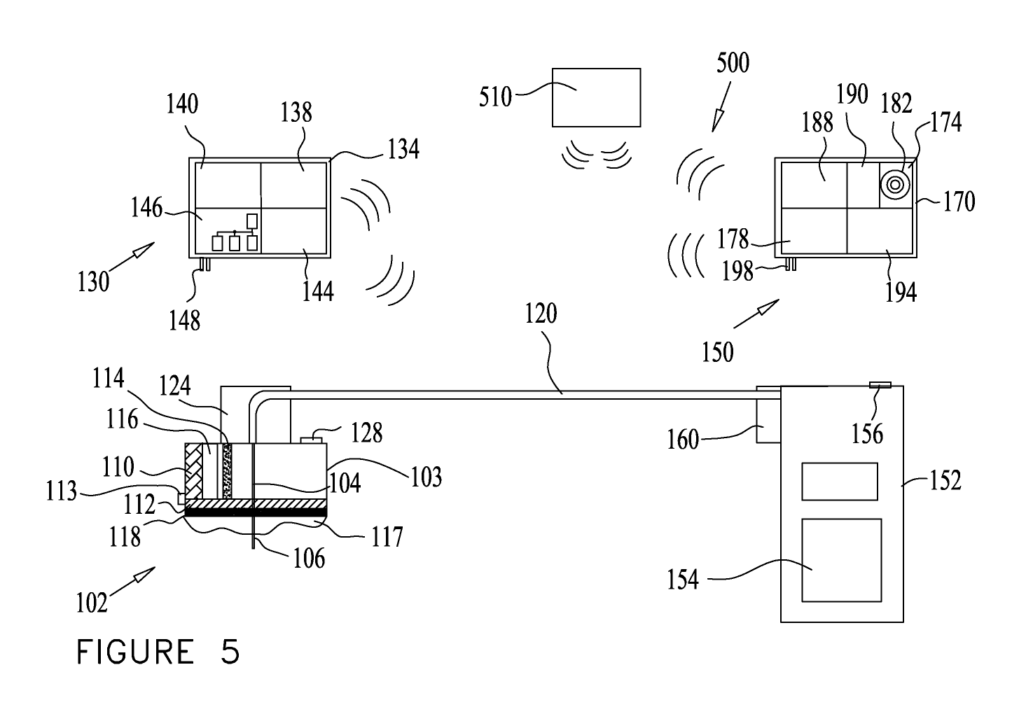

FIG. 5 is a schematic illustration of an exemplary therapeutic fluid delivery system 500, according to some embodiments of the present disclosure. The therapeutic fluid delivery system 500 can include one or more of the components and/or combination of components of therapeutic fluid delivery systems 100, 300, and 400, as shown in FIGS. 1-4. For discussion purposes only, FIG. 5 illustrates system 100 shown in FIG. 1. The system 500 can include an external unit 510 that can receive signals from the catheter adaptor 130, and/or the pump adaptor 150, and/or the adaptor 360 (as shown in FIGS. 3 and 4).

In some embodiments, the system 500 can include an external unit 510. The external unit 510 can include at least one of the following: a personal computer, a laptop, a cellular telephone, a smartphone, a tablet, a media player, a personal digital assistant (“PDA”), a storage unit (e.g., a database), a server, and/or any other computing device, and/or any combination thereof. In some embodiments, the external unit 510 can include a glucose meter. The database can include any suitable device that can store data and/or perform analysis thereof. The database can include a processor and/or memory.

The external unit 510 can include a processor for processing the detected signal received from the catheter adaptor 130 and/or the pump adaptor 150 and/or the adaptor 360 (as shown in FIGS. 3 and 4). A memory can also be provided for storing the detected signal.

The detected signal can be processed to generate data related to the activity of the infusion set 102, such as an amount of injected drug, an injection time, a duration of injection, and/or any other information. The data can comprise information related to the type of drug as well as identification of the infusion set 102 and/or the pump 152. In exemplary embodiments, where treatment of a diabetic patient is performed, different types and/or quantities of insulin can be administered based on a basal insulin dose and/or a bolus insulin dose, where different doses can be injected using different infusion sets 102. Thus, data indicating type of injected dose and/or infusion set can assist a user, a caretaker and/or a physician monitoring the course of treatment. In some embodiments, the data can include date and time of an injection.

In some embodiments, the data can be used to monitor expiration of the drug. This can be determined by measuring the time discrepancy between a first use of the infusion set 102 and a current time and/or by comparing the date of the drug infusion with an expiration date provided by the drug manufacturer.

The data can be used by a physician, a caretaker and/or the patient to track treatment goals. Further, the data can be analyzed together with data provided by a glucose meter (e.g., such as, external unit 510 shown in FIG. 5). Additionally, the data can be used to alert the patient if the drug that is to be injected into the patient has surpassed its expiration date. Further, the data can be used to alert the patient upon reduction of the efficacy of the drug due to excess heat or any other relevant parameter.

In some embodiments, the treatment element 112 can include a treatment device disclosed in co-owned International Patent Application No. PCT/IB2008/051044, the disclosure of which is incorporated herein by reference in its entirety.

Various implementations of some of embodiments disclosed, in particular at least some of the processes discussed (or portions thereof), may be realized in digital electronic circuitry, integrated circuitry, specially configured ASICs (application specific integrated circuits), computer hardware, firmware, software, and/or combinations thereof. These various implementations, such as associated with the drug dispensing-tracking system 100, 300 or 400 and the components thereof, for example, may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Such computer programs (also known as programs, software, software applications or code) include machine instructions/code for a programmable processor, for example, and may be implemented in a high-level procedural and/or object-oriented programming language, and/or in assembly/machine language. As used herein, the term “machine-readable medium” refers to any computer program product, apparatus and/or device (e.g., non-transitory mediums including, for example, magnetic discs, optical disks, flash memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a LCD (liquid crystal display) monitor and the like) for displaying information to the user and a keyboard and/or a pointing device (e.g., a mouse or a trackball, touchscreen) by which the user may provide input to the computer. For example, this program can be stored, executed and operated by the dispensing unit, remote control, PC, laptop, smartphone, media player or personal data assistant (“PDA”). Other kinds of devices may be used to provide for interaction with a user as well. For example, feedback provided to the user may be any form of sensory feedback (e.g., visual feedback, auditory feedback, or tactile feedback), and input from the user may be received in any form, including acoustic, speech, or tactile input. Certain embodiments of the subject matter described herein may be implemented in a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components.

The components of the system may be interconnected by any form or medium of digital data communication (e.g., a communication network). Examples of communication networks include a local area network (“LAN”), a wide area network (“WAN”), and the Internet. The computing system according to some such embodiments described above may include clients and servers. A client and server are generally remote from each other and typically interact through a communication network. The relationship of client and server arises by virtue of computer programs running on the respective computers and having a client-server relation to each other.

Any and all references to publications or other documents, including but not limited to, patents, patent applications, articles, webpages, books, etc., presented anywhere in the present application, are herein incorporated by reference in their entirety.

Example embodiments of the devices, systems and methods have been described herein. As may be noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements/features from any other disclosed methods, systems, and devices, including any and all features corresponding to translocation control. In other words, features from one and/or another disclosed embodiment may be interchangeable with features from other disclosed embodiments, which, in turn, correspond to yet other embodiments. Furthermore, one or more features/elements of disclosed embodiments may be removed and still result in patentable subject matter (and thus, resulting in yet more embodiments of the subject disclosure).

1. A system for delivering a therapeutic fluid into a tissue comprising:
an infusion set comprising a catheter for delivery of a dose of the therapeutic fluid into the tissue via an infusion tube;
a treatment element configured to apply a treatment to the tissue proximate to the catheter;
a catheter adaptor comprising a first transponder; and
a pump adaptor in communication with the catheter adaptor and comprising:
a second transponder configured to communicate with the first transponder, and
an infusion detection sensor configured to detect an infusion of the therapeutic fluid;
wherein, upon detection of an infusion of the therapeutic fluid by the infusion detection sensor, the second transponder is configured to communicate a signal indicative of the detected infusion to the first transponder, wherein the catheter adaptor is configured to cause the treatment element to apply treatment to the tissue proximate to the catheter based on at least one of the following: the signal indicative of the detected infusion and a manual activation of the catheter adaptor causing the treatment element to apply treatment, wherein at least one of a strength and a duration of the treatment is determined based on a dose of the infused therapeutic fluid.

2. The system according to claim 1, wherein the treatment element applies treatment during at least one of the following times: before infusion of the therapeutic fluid, during infusion of the therapeutic fluid, and after infusion of the therapeutic fluid.

3. The system according to claim 1, wherein at least one of the first transponder and the second transponder includes at least one of the following: a wireless transponder, and a radio frequency identification device (RFID), an antenna, a transducer, and/or an RLC circuit, an electrical circuit for analog or digital short range communication, or a digital communication mode including WIFI or Bluetooth.

4. The system according to claim 1, wherein the pump adaptor is connected to a pump and a reservoir containing the therapeutic fluid, wherein the pump is configured to pump the therapeutic fluid from the reservoir to the catheter via the infusion tube.

5. The system according to claim 4, wherein the infusion detection sensor is configured to detect an amount of the therapeutic fluid being pumped by the pump based on at least one movement of the pump.

6. (canceled)

7. (canceled)

8. The system according to claim 1, wherein the dose of the therapeutic fluid being infused is at or above a predetermined dose.

9. The system according to claim 1, wherein the therapeutic fluid includes at least one of the following: subcutaneously delivered therapeutic fluids, insulin, rapid-acting insulin, insulin mimetics, insulin analogs, and/or any other types of insulin, pain relief drugs or cancer treatment drugs.

10. (canceled)

11. The system according to claim 1, wherein:
the pump adaptor includes:
a pump adaptor power source;
a pump adaptor controller; and
wherein the controller, upon receiving an indication from the infusion detection sensor, instructs the second transponder to communicate with the first transponder; and
the catheter adaptor includes:
a catheter adaptor power source;
a catheter adaptor controller; and
at least one first electrical contact configured to be coupled with at least one second electrical contact disposed on the infusion set;
wherein the catheter adaptor controller is configured to process the communication received from the second transponder and generate an instruction to the treatment element to initiate the application of treatment, the generated instruction being provided to the treatment element using the at least one first electrical contact and the at least one second electrical contact.

12. The system according to claim 1, wherein the application of treatment includes at least one of the following: heating, cooling, mechanical vibrations, suction, massaging, acoustic stimulation, electromagnetic radiation, magnetic stimulation, radio frequency irradiation, microwave irradiation, electrical stimulation, Transcutaneous Electrical Nerve Stimulation (TENS), an additional substance, drugs, medicament, chemicals, biologically active bacteria, biologically inactive bacteria or a combination thereof.

13. (canceled)

14. The system according to claim 1, wherein the application of treatment is configured to modify a pharmacokinetic and/or a pharmacodynamic profile of the therapeutic fluid being infused.

15. A method for delivering a therapeutic fluid comprising:
providing a system according to claim 1;
initiating infusion of the therapeutic fluid using an infusion pump;
detecting the initiation of the infusion of the therapeutic fluid using the infusion detection sensor;
generating a communication to the first transponder using the second transponder, wherein the communication comprises a signal indicative of the detected infusion; and
activating the treatment element using the catheter adaptor to apply treatment in accordance with a dose of the therapeutic fluid being infused.

16. The method according to claim 15, wherein treatment is applied during at least one of the following times: before infusion of the therapeutic fluid, during infusion of the therapeutic fluid, and after infusion of the therapeutic fluid.

17. The method according to claim 15, wherein at least one of the first transponder and the second transponder includes at least one of the following: a wireless transponder, and a radio frequency identification device (RFID), an antenna, a transducer, and/or an RLC circuit, an electrical circuit for analog or digital short range communication, or a digital communication mode including WIFI or Bluetooth.

18. The method according to claim 15,
wherein the pump adaptor is connected to a pump and a reservoir containing the therapeutic fluid, and
wherein the initiating comprises pumping the therapeutic fluid from the reservoir to the catheter via the infusion tube.

19. The method according to claim 18, wherein the detecting includes detecting an amount of the therapeutic fluid being pumped by the pump based on at least one movement of the pump.

20. (canceled)

21. (canceled)

22. The method according to claim 15, wherein the dose of the therapeutic fluid being infused is at or above a predetermined dose.

23. The method according to claim 15, wherein the therapeutic fluid includes at least one of the following: subcutaneously delivered therapeutic fluids, insulin, rapid-acting insulin, insulin mimetics, insulin analogs, and/or any other types of insulin, pain relief drugs or cancer treatment drugs.

24. (canceled)

25. The method according to claim 15, wherein
the pump adaptor includes:
a pump adaptor power source;
a pump adaptor controller; and
wherein the generating further comprises receiving, using the pump adaptor controller, an indication from the infusion detection sensor and instructing, using the pump adaptor controller, the second transponder to communicate with the first transponder; and
the catheter adaptor includes
a catheter adaptor power source;
a catheter adaptor controller; and
at least one first electrical contact configured to couple with at least one second electrical contact disposed on the infusion set;
wherein the catheter adaptor controller is configured to process the communication received from the second transponder and generate an instruction to the treatment element to apply treatment, the generated instruction being provided to the treatment element using the at least one first electrical contact and the at least one second electrical contact.

26. The method according to claim 15, wherein the application of treatment includes at least one of the following: heating, cooling, mechanical vibrations, suction, massaging, acoustic stimulation, electromagnetic radiation, magnetic stimulation, radio frequency irradiation, microwave irradiation, electrical stimulation, Transcutaneous Electrical Nerve Stimulation (TENS), an additional substance, drugs, medicament, chemicals, biologically active bacteria, biologically inactive bacteria or a combination thereof.

27. (canceled)

28. The method according to claim 15, wherein the application of treatment is configured to modify a pharmacokinetic and/or a pharmacodynamic profile of the therapeutic fluid being infused.

29. (canceled)

30. (canceled)

31. (canceled)
